(19) United States
(12) Patent Application Publication
(10) Pub. No.: US 2019/0133931 A1
(43) Pub. Date: May 9, 2019

(54) SUBCONJUNCTIVAL DEPOT FORMING FORMULATIONS FOR OCULAR DRUG DELIVERY

(71) Applicant: Nanyang Technological University, Singapore (SG)

(72) Inventors: Subramanian VENKATRAMAN, Singapore (SG); Rini Rachel JOSEPH, Singapore (SG); Yin Chiang Freddy BOEY, Singapore (SG)

(21) Appl. No.: 16/095,269

(22) PCT Filed: Apr. 19, 2017
(86) PCT No.: PCT/SG2017/050217

§ 371 Date: Oct. 19, 2018

(30) Foreign Application Priority Data
Apr. 19, 2016 (SG) 10201603076T

(51) Int. Cl. A61K 9/00; A61K 9/127

(52) U.S. Cl. CPC A61K 9/0051; A61K 9/127; A61K 9/1277; A61K 9/0048

(57) ABSTRACT

According to the present disclosure, the use of a liposomal formulation comprising one or more phospholipids in the manufacture of a medicament, or in a method for the treatment of posterior and/or anterior ocular segment diseases is provided. Preferred phospholipids include POPC and DOTAP, POPC and POPG, DPTAP and POPG, DMTAP and POPG, DPPC and DPTAP, DPPC and DPPG, DMPC and DMTAP, or DMPC and DMPG. In a separate embodiment, the use of a particulate formulation comprising a plurality of poly(lactic-co-glycolic acid) particles in the manufacture of a medicament, or in a method for the treatment of posterior and/or anterior ocular segment diseases is also provided. In either embodiments, the posterior ocular segment diseases comprise age related degeneration, diabetic macular edema or retinopathy and anterior ocular segment diseases comprise glaucoma, cataract or uveitis.

### CROSS-REFERENCE TO RELATED APPLICATION

This application claims the benefit of priority of Singapore Patent Application No. 10201603076T, filed 19 Apr. 2016, the content of it being hereby incorporated by reference in its entirety for all purposes.

### TECHNICAL FIELD

The present disclosure relates to a liposomal or particulate formulation and its use in the treatment of posterior and/or anterior ocular segment diseases.

### BACKGROUND

Several diseases of the posterior ocular segment are conventionally treated by administering the medications via intravitreal injection. As this is an invasive procedure which may damage the integrity of the eyeball, the injection procedure itself may lead to several sight-threatening complications such as endophthalmitis, cataract or even retinal detachment.

Meanwhile, topical administration of drugs has shown very little promise in the treatment of posterior segment diseases due to tear washout, lacrimal drainage and the presence of various ocular anatomical barriers, thereby resulting in less than 5% bioavailability of the drug at the site of action.

Systemic administration may be ineffective due to the presence of the blood-ocular barrier which limits access to the disease-site. The large quantities of drug required for systemic administration also tend to cause undesirable systemic side effects.

Due to issues associated with the various modes of administration as mentioned above, other local modes of administration have been developed in recent times to improve retinal delivery of drugs. These techniques involve administration of drugs periocularly which then diffuse across tissue barriers to reach the site of action. In this regard, the main challenge to periocular delivery is often the transport of drug(s) across the tissue barriers to reach the required site of action. Transport characteristics of several drugs and macromolecules across the sclera have been explored over the past five decades to study on the factors that affect transport across this barrier. Several factors may influence the transport of drugs to the posterior ocular segment. Bearing in mind that such factors exist and considering that many diseases affecting the posterior segment may be chronic in nature, it may be desirable to develop sustained release systems for periocular (e.g. subconjunctival) administration in order to extend their duration of action and to reduce the frequency of injections. Various attempts have thus been made to improve on the therapeutic effect of drugs to the posterior segment via the use of sustained release systems such as inserts, implants, micro and nano particulate systems etc.

Particularly, subconjunctival injection of microparticles and nanoparticles in a rat model has been studied and demonstrated effective sustained delivery to the retina. In another published study, transport of rigid polycarbonate nanoparticles of different sizes in vivo was investigated. It was observed that these particles, even at the smallest size studied (20 nm), were unable to achieve significant transport across the sclera even though particles of 200 nm and 2 μm were retained at the site of action for more than 2 months. In another study, comparisons were made between the transport of bare drugs against drugs encapsulated in poly(lactic-co-glycolic acid) (PLGA) nanoparticles as well as liposomes, in both ex vivo and in vivo setups, and based on the amount of drugs recovered from the scleral washout, it was indirectly concluded that the carriers do not cross the sclera.

Despite the above, various efforts including the studies as mentioned above, did not investigate the properties of the nanocarriers which can enable them to achieve transport across the sclera or act as depots, and how the properties can be tuned.

Based on the above, there is thus a need to provide for a formulation that ameliorates one or more of the drawbacks as mentioned above. There is also a need to provide for a method of treating ocular diseases which addresses one or more of the above issues.

### SUMMARY

In one aspect, the present disclosure provides for the use of a liposomal formulation comprising one or more phospholipids in the manufacture of a medicament for the treatment of posterior and/or anterior ocular segment diseases, wherein the one or more phospholipids form at least one liposome each comprising at least one phospholipid bilayer, and wherein the one or more phospholipids comprise 1-palmitoyl-2-oleoyl-sn-glycero-3-phosphocholine (POPC), 1,2-dihexadecanoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 1,2-dipalmitoyl-3-trimethylammonium-propane (DPTAP), 1,2-dimyristoyl-3-trimethylammonium-propane (DMTAP), 1,2-dioleoyl-3-trimethylammonium-propane (DOTAP), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1-palmitoyl-2-oleoyl-sn-glycero-3-phosphoglycerol (POPG), hydrogenated soybean phosphatidylcholine (HSPC), 1-stearoyl-2-oleoyl-sn-glycero-3-phosphocholine (SOPC), or their combination thereof.

In another aspect, the present disclosure provides for a liposomal formulation comprising one or more phospholipids for use in the treatment of posterior and/or anterior ocular segment diseases, wherein the one or more phospholipids form at least one liposome each comprising at least one phospholipid bilayer, and wherein the one or more phospholipids comprise 1-palmitoyl-2-oleoyl-sn-glycero-3-phosphocholine POPC), 1,2-dihexadecanoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 1,2-dipalmitoyl-3-trimethylammonium-propane (DPTAP), 1,2-dimyristoyl-3-trimethylammonium-propane (DMTAP), 1,2-dioleoyl-3-trimethylammonium-propane (DOTAP), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1-palmitoyl-2-oleoyl-sn-glycero-3-phosphoglycerol (POPG), hydrogenated soybean phosphatidylcholine (HSPC), 1-stearoyl-2-oleoyl-sn-glycero-3-phosphocholine (SOPC), or their combination thereof.

In another aspect, the present disclosure provides for a method of treating posterior and/or anterior ocular segment diseases by administering a liposomal formulation comprising one or more phospholipids, wherein the one or more phospholipids form at least one liposome each comprising at least one phospholipid bilayer, and wherein the one or more phospholipids comprise 1-palmitoyl-2-oleoyl-sn-glycero-3-phosphocholine (POPC), 1,2-dihexadecanoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 1,2-dipalmitoyl-3-trimethylammonium-propane (DPTAP), 1,2-dimyristoyl-3-trimethylammonium-propane (DMTAP), 1,2-dioleoyl-3-trimethylammonium-propane (DOTAP), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1-palmitoyl-2-oleoyl-sn-glycero-3-phosphoglycerol (POPG), hydrogenated soybean phosphatidylcholine (HSPC), 1-stearoyl-2-oleoyl-sn-glycero-3-phosphocholine (SOPC), or their combination thereof.

In another aspect, the present disclosure provides for the use of a particulate formulation comprising a plurality of poly(lactic-co-glycolic acid) (PLGA) particles in the manufacture of a medicament for the treatment of posterior and/or anterior ocular segment diseases, wherein the plurality of PLGA particles comprise PLGA microparticles and/or PLGA nanoparticles.

In another aspect, the present disclosure provides for a particulate formulation comprising a plurality of poly(lactic-co-glycolic acid) (PLGA) particles for use in the treatment of posterior and/or anterior ocular segment diseases, wherein the plurality of PLGA particles comprise PLGA microparticles and/or PLGA nanoparticles.

In another aspect, the present disclosure provides for a method of treating posterior and/or anterior ocular segment diseases by administering a particulate formulation comprising a plurality of poly(lactic-co-glycolic acid) (PLGA) particles, wherein the plurality of PLGA particles comprise PLGA microparticles and/or PLGA nanoparticles.

### BRIEF DESCRIPTION OF THE DRAWINGS

In the drawings, like reference characters generally refer to like parts throughout the different views. The drawings are not necessarily to scale, emphasis instead generally being placed upon illustrating the principles of the invention. In the following description, various embodiments of the present disclosure are described with reference to the following drawings, in which:

FIG. 1 shows a schematic representation of the subconjunctival route compared with the intravitreal route.

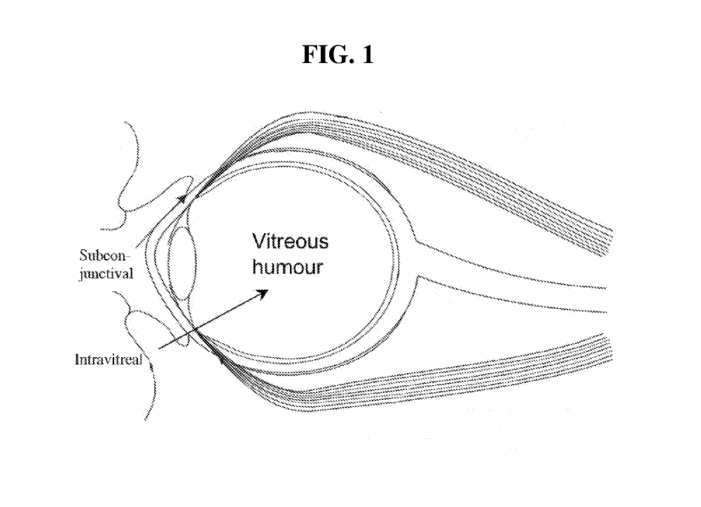

FIG. 2a shows a representative epifluorescence micrograph of a sclera post transport experiment with 1-palmitoyl-2-oleoyl-sn-glycero-3-phosphocholine (POPC) liposomes of the various embodiments as disclosed herein. The POPC liposomes are fluorescent labelled. Specifically, POPC multilamellar vesicles (MLVs) of a larger size (about 1 μm) are found to form episcleral depot(s) without penetrating into the sclera. The liposomes are labelled with rhodamine-PE (red). The edges of the tissue are marked with a dotted line for clarity. The scale bar represents 100 μm.

FIG. 2b shows a representative epifluorescence micrograph of a sclera post transport experiment with POPC liposomes of the various embodiments as disclosed herein. The POPC liposomes are fluorescent labelled. Specifically, large POPC unilamellar vesicles (LUVs) of 90 nm size are found to form intrascleral depot(s) which penetrated the sclera to some extent. The liposomes are labelled with rhodamine-PE (red). The edges of the tissue are marked with a dotted line for clarity. The scale bar represents 100 μm.

FIG. 2c shows a representative epifluorescence micrograph of a sclera post transport experiment with POPC liposomes of the various embodiments as disclosed herein. The POPC liposomes are fluorescent labelled. Specifically, the POPC LUVs of 70 nm size exhibited a larger intrascleral depot effect with higher penetration into the sclera. The liposomes are labelled with rhodamine-PE (red). The edges of the tissue are marked with a dotted line for clarity. The scale bar represents 100 μm.

FIG. 3a shows a representative epifluorescent micrograph of the sclera post transport experiment wth POPC liposomes of the various embodiments as disclosed herein. The POPC liposomes are fluorescent labelled. Specifically, POPC liposomes, being more fluid, have the ability to act as intrascleral depots compared to 1,2-dihexadecanoyl-sn-glycero-3-phosphocholine (DPPC) liposomes of FIG. 3b which have a higher transition temperature. The POPC liposomes are labelled with rhodamine-PE (red) and the edges of the tissue are marked with a dotted line for clarity. The scale bar represents 100 μm.

FIG. 3b shows a representative epifluorescent micrograph of the sclera post transport experiment wth DPPC liposomes of the various embodiments as disclosed herein. The DPPC liposomes are fluorescent labelled. These DPPC liposomes having a higher transition temperature were found to transport less into the sclera. The liposomes are labelled with rhodamine-PE (red) and the edges of the tissue are marked with a dotted line for clarity. The scale bar represents 100 μm.

FIG. 3c shows a representative epifluorescent micrograph of the sclera post transport experiment wth 1,2-dioleoyl-sn-glycero-3-phosphocholine (DOPC) liposomes of the various embodiments as disclosed herein. The DOPC liposomes are fluorescent labelled. The liposomes are labelled with rhodamine-PE (red) and the edges of the tissue are marked with a dotted line for clarity. The scale bar represents 100 μm.

FIG. 4a shows a representative epifluorescent micrograph of the sclera post transport experiment (ex vivo setup) with POPC (neutral) liposomes of the various embodiments as disclosed herein. The POPC liposomes are fluorescently labelled. The POPC liposomes formed intrascleral depots and demonstrated transport into the sclera. The liposomes are labelled with rhodamine-PE (red) and the edges of the tissue are marked with a dotted line for clarity. The scale bar represents 100 μm.

FIG. 4b shows a representative epifluorescent micrograph of the sclera post transport experiment (ex vivo setup) with POPC-DOTAP (positively charged) liposomes of the various embodiments as disclosed herein. DOTAP refers to 1,2-dioleoyl-3-trimethylammonium-propane. The POPC-DOTAP liposomes are fluorescent labelled. The POPC-DOTAP (positively charged) liposomes formed episcleral depots and were stuck to the episcleral surface. The liposomes are labelled with rhodamine-PE (red) and the edges of the tissue are marked with a dotted line for clarity. The scale bar represents 100 μm.

FIG. 4c shows a representative epifluorescent micrograph of the sclera post transport experiment (ex vivo setup) with POPC-POPG (negatively charged) liposomes of the various embodiments as disclosed herein. POPG refers to 1-palmitoyl-2-oleoyl-sn-glycero-3-phosphoglycerol. The POPC-POPG liposomes are fluorescent labelled. The POPC-POPG (negatively charged) liposomes are found to exhibit a minor degree of transport into the sclera specifically for an ex vivo setup. The liposomes are labelled with rhodamine-PE (red) and the edges of the tissue are marked with a dotted line for clarity. The scale bar represents 100 μm.

FIG. 5a shows a representative epifluorescent micrograph of the sclera post transport experiment (ex vivo setup) based on unsaturated POPC liposomes without cholesterol according to embodiments as disclosed herein. The POPC liposomes are fluorescent labelled. Specifically, POPC (neutral) liposomes without cholesterol formed intrascleral depots and demonstrated transport into the sclera. The liposomes are labelled with rhodamine-PE (red) and the edges of the tissue are marked with a dotted line for clarity. The scale bar represents 100 μm.

FIG. 5b shows a representative epifluorescent micrograph of the sclera post transport experiment (ex vivo setup) based on unsaturated POPC liposomes with addition of cholesterol according to embodiments as disclosed herein. The POPC liposomes are fluorescent labelled. Specifically, POPC-Cholesterol liposomes in a 80:20 molar ratio exhibited a lesser degree of transport into the sclera. The liposomes are labelled with rhodamine-PE (red) and the edges of the tissue are marked with a dotted line for clarity. The scale bar represents 100 μm.

FIG. 6a shows a representative epifluorescent micrograph of the sclera post transport experiment based on saturated DPPC liposomes without cholesterol according to embodiments as disclosed herein. The DPPC liposomes are fluorescent labelled. Specifically, the DPPC (neutral) liposomes without cholesterol penetrated the sclera to a lesser extent compared to the DPPC-Cholesterol liposomes of FIG. 6b. The liposomes are labelled with rhodamine-PE (red) and the edges of the tissue are marked with a dotted line for clarity. The scale bar represents 100 μm.

FIG. 6b shows a representative epifluorescent micrograph of the sclera post transport experiment based on saturated DPPC liposomes with addition of cholesterol (80:20 molar ratio of DPPC:Cholesterol) according to embodiments as disclosed herein. The DPPC liposomes are fluorescent labelled. Specifically, the DPPC (neutral) liposomes with cholesterol penetrated the sclera to a higher extent compared to FIG. 6a. The liposomes are labelled with rhodamine-PE (red) and the edges of the tissue are marked with a dotted line for clarity. The scale bar represents 100 μm.

FIG. 7a shows a representative epifluorescence micrograph of PLGA microparticles of 75 μm after 96 hours of transport. The PLGA particles are tagged with fluoresceinamine (green). The edges of the scleral tissue have been marked with a dotted line for clarity. The scale bar represents 100 μm.

FIG. 7b shows a representative epifluorescence micrograph of PLGA nanoparticles of about 200 nm after 96 hours of transport. The PLGA particles are tagged with fluoresceinamine (green). The edges of the scleral tissue have been marked with a dottted line for clarity. The scale bar represents 100 μm.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and changes may be made without departing from the scope of the invention. The various embodiments are not necessarily mutually exclusive, as some embodiments can be combined with one or more other embodiments to form new embodiments.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

With the above in mind, the present disclosure provides for a liposomal formulation and/or a particulate formulation, and their use in the treatment and/or manufacture of a medicament for the treatment of posterior and/or anterior ocular segment diseases. The present disclosure also relates to a method of treating such ocular segment diseases using the liposomal formulation and/or the particulate formulation. Embodiments described in the context of the liposomal or particulate formulation and their uses are analogously valid for the method of treating as described herein, and vice versa.

Before going into the details of the liposomal and particulate formulations, their uses, the method of treating such ocular diseases using the liposomal formulation or the particulate formulation, and the various embodiments, the definitions of certain terms, expressions or phrases are first discussed.

The word “substantially” does not exclude “completely” e.g. a composition which is “substantially free” from Y may be completely free from Y. Where necessary, the word “substantially” may be omitted from the definition of the invention.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance.

As used herein, the term “and/or” includes any and all combinations of one or more of the associated listed items.

As used herein, the phrase of the form of “at least one of A and B” may include A or B or both A and B. Correspondingly, the phrase of the form of “at least one of A and B and C”, or including further listed items, may include any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements. Meanwhile, the terms “consisting” and “consist”, and grammatical variants thereof, are intended to represent “close” or “exclusive” language such that they solely include the recited elements but exclude additional, unrecited elements.

Having defined the various terms, expressions and phrases, details of the liposomal and particulate formulations and their uses, the method of treating and the various embodiments are now described below.

In the present disclosure, the use of a liposomal formulation comprising one or more phospholipids in the manufacture of a medicament for the treatment of posterior and/or anterior ocular segment diseases is disclosed. The one or more phospholipids may form at least one liposome each comprising at least one phospholipid bilayer, and the one or more phospholipids may comprise or may consist of 1-palmitoyl-2-oleoyl-sn-glycero-3-phosphocholine (POPC), 1,2-dihexadecanoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 1,2-dipalmitoyl-3-trimethylammonium-propane (DPTAP), 1,2-dimyristoyl-3-trimethylammonium-propane (DMTAP), 1,2-dioleoyl-3-trimethylammonium-propane (DOTAP), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1-palmitoyl-2-oleoyl-sn-glycero-3-phosphoglycerol (POPG), hydrogenated soybean phosphatidylcholine (HSPC), 1-stearoyl-2-oleoyl-sn-glycero-3-phosphocholine (SOPC), or their combination thereof. The one or more phospholipids present in combination may be any positively or negatively charged phospholipids. Non-limiting examples of the combination may include POPC and DOTAP, POPC and POPG, DPTAP and POPG, DMTAP and POPG, DPPC and DPTAP, DPPC and DPPG, DMPC and DMTAP, or DMPC and DMPG.

In various embodiments disclosed throughout the present disclosure, POPC, DPPC and DMPC may constitute the base lipids. Other suitable lipids may also be used as the base lipids.

In some embodiments, the one or more phospholipids may have a transition temperature of −18° C. to 55° C. or other any temperature or range within this specified range. In the context of the present disclosure, the phrase “transition temperature” refers to the temperature required to induce a change in the phospholipid from an ordered gel phase where the hydrocarbon chains are fully extended and closely packed, to a disordered liquid phase where the hydrocarbon chains are randomly oriented and fluid (i.e. the structure of phospholipids allows better diffusion in and out of the liposome(s) compared to one that is rigid). A higher transition temperature means that the phospholipid requires a higher temperature to become more fluid. Advantageously, phospholipids having these transition temperatures possess the ability to form episcleral or intrascleral depot(s). The term “depot” as used herein refers to a body area in which a substance, e.g. a pharmceutical composition, drug or any other therapeutic agent, can be accumulated, deposited or stored and from which it can be distributed.

In various embodiments, the one or more phospholipids may carry a net positive charge, a net negative charge or a net neutral charge. The phrase “neutral charge” refers to a phospholipid or a liposome that contains neither an overall (i.e. net) positive charge nor overall negative charge. To illustrate on this, POPC is used as a non-limiting example. The structure of POPC is shown as follows.

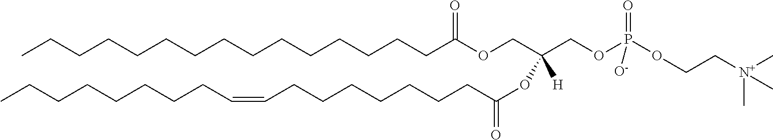 [SMILES: CCCCCCCC/C=C\CCCCCCCC(=O)O[C@H](COC(=O)CCCCCCCCCCCCCCC)COP(=O)([O-])OCC[N+](C)(C)C]

In POPC, the net charge is neutral because the negative charge of the phosphate group is neutralized by the quaternary ammonium group that is positively charged. In contrast, DOTAP as depicted below carries only the positive charged quaternary ammonium group and hence the net charge of DOTAP becomes positive.

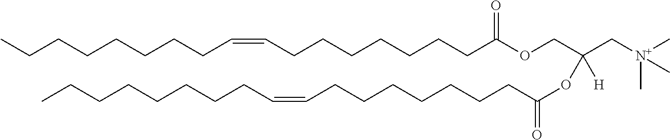 [SMILES: CCCCCCCC/C=C\CCCCCCCC(=O)OCC(C[N+](C)(C)C)OC(=O)CCCCCCC/C=C\CCCCCCCC]

The net charge of the one or more phospholipids may depend on its combination with other entities such as lipids, any molecule(s) or atom(s) etc. For example, when a neutral phospholipid such as POPC is combined with DOTAP, the combined phospholipid becomes positively charged.

As the net charge of the phospholipid may be imparted onto the liposome, it may influence the liposomes' depot forming ability based on the location at which the depot is formed. For instance, the sclera is negatively charged and thus may retain liposomes that comprise positively charged phospholipids, thereby forming episcleral depots instead of intrascleral depots. Hence, the formulation is advantageously versatile as it can be tailored to form different types of depots based on the charge of the tissue surface or body (e.g. ocular) area.

Apart from the net charge of the phospholipid, the one or more phospholipids may comprise a saturated or an unsaturated phospholipid. The term “saturated” means that the phospholipids do not contain carbon-carbon double and triple bonds. Meanwhile, the term “unsaturated” means that the phospholipids may contain one or more carbon-carbon double and/or triple bonds. The degree of saturation may need to be considered. This is because degree of penetration of the depots into the sclera may depend on the degree of saturation of the phospholipids in the presence or absence of other components. In this regard, cholesterol may be present in the liposomes or added/combined with the one or more phospholipids. When cholesterol is present with unsaturated lipids, the at least one phospholipid bilayer of the liposomes may become more rigid which in turn results in lower penetration into the sclera. When cholesterol is present with saturated lipids, the cholesterol may destabilize the bilayer, causing the liposome to become leakier to result in higher degree of penetration. Accordingly, the at least one liposome comprising the saturated or unsaturated phospholipid may further comprise cholesterol. In other instances, the at least one liposome comprising the saturated or unsaturated phospholipid may not comprise or contain cholesterol.

In various embodiments, at least one liposome may be a multilamellar vesicle or a unilamellar vesicle. Multilamellar liposomes may be composed of a plurality of concentric phospholipid bilayers comprising the one or more phospholipids while unilamellar liposomes may be a vesicle having a single bilayer of the one or more phospholipids. The vesicle may be used to encapsulate any suitable pharmaceutical compositions, drugs or therapeutic agents for treating ocular diseases, such as posterior and/or anterior ocular segment diseases. This implies that the liposomes formed from the one or more phospholipids may be taken as liposomal microparticles or liposomal nanoparticles acting as carriers (e.g. microcarriers or nanocarriers) to deliver suitable drugs for treating ocular diseases. Hence, the liposomal formulation may be known as microparticle(s) or nanoparticle(s) formulation in the present disclosure. The liposomes may be spherical or substantially spherical. It need not be a perfect sphere as the liposomes may change its shape when it penetrates the sclera or other ocular tissues.

In various embodiments, the at least one liposome may have a size of 30 nm to 2 μm, 30 nm to 1 μm, 100 nm to 1 μm, 500 nm to 1 μm, 30 nm to 100 nm, 30 nm to 500 nm, 100 nm to 500 nm or any other sizes or ranges falling within these ranges. In various embodiments, the size may be 100 nm to 500 nm when the transition temperature of the one or more phospholipids may be 37° C. or more.

As mentioned earlier, the formulation may be formed as an episcleral depot or an intrascleral depot according to the embodiments disclosed herein. Factors that influence this outcome have been discussed above and/or illustrated in the examples.

Regardless of the type of depot formed, the liposomal formulation can be used to treat or used in the manufacture of a medicament for the treatment of various ocular diseases. Such ocular diseases may include but are not limited to posterior ocular segment diseases and/or anterior ocular segment diseases. The earlier may comprise age-related macular degeneration (AMD), diabetic macular edema (DME), diabetic retinopathy etc. while the latter may comprise glaucoma, cataract, uveitis etc.

The present disclosure also relates to a liposomal formulation comprising one or more phospholipids for use in the treatment of posterior and/or anterior ocular segment diseases, wherein the one or more phospholipids form at least one liposome each comprising at least one phospholipid bilayer, and wherein the one or more phospholipids comprise 1-palmitoyl-2-oleoyl-sn-glycero-3-phosphocholine (POPC), 1,2-dihexadecanoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 1,2-dipalmitoyl-3-trimethylammonium-propane (DPTAP), 1,2-dimyristoyl-3-trimethylammonium-propane (DMTAP), 1,2-dioleoyl-3-trimethylammonium-propane (DOTAP), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1-palmitoyl-2-oleoyl-sn-glycero-3-phosphoglycerol (POPG), hydrogenated soybean phosphatidylcholine (HSPC), 1-stearoyl-2-oleoyl-sn-glycero-3-phosphocholine (SOPC), or their combination thereof. The one or more phospholipids present in combination may be any positively or negatively charged phospholipids. Non-limiting examples of the combination may include POPC and DOTAP, POPC and POPG, DPTAP and POPG, DMTAP and POPG, DPPC and DPTAP, DPPC and DPPG, DMPC and DMTAP, or DMPC and DMPG. Accordingly, the liposomal formulation may be used in treating posterior and/or anterior ocular segment diseases.

In various embodiments, the one or more phospholipids may have a transition temperature of −18° C. to 55° C. or other any temperature or range within this specified range.

In various embodiments, the one or more phospholipids may carry a net positive charge, a net negative charge or a net neutral charge. The net charge of the one or more phospholipids may depend on its combination with entities such as lipids, any molecule(s) or atom(s) etc. The present liposomal formulation is advantageously versatile as the charge of the phopholipids, and hence the liposomes, can be tailored based on the tissue surface or body (e.g. ocular) area as mentioned above.

The one or more phospholipids may comprise or consist of a saturated or an unsaturated phospholipid according to various embodiments. The saturated or unsaturated phospholipid(s) may be combined with entities such as lipids, any molecule(s) or atom(s) etc. In this regard, the saturated or unsaturated phospholipid(s) may further comprise or consist of cholesterol. Alternatively, the saturated or unsaturated phospholipid(s) may not comprise or consist of cholesterol. Accordingly, the at least one liposome comprising the saturated or unsaturated phospholipid(s) may further comprise cholesterol. In other instances, the at least one liposome comprising the saturated or unsaturated phospholipid(s) may not comprise or contain cholesterol. The effects of cholesterol on the phospholipid(s), and consequently the liposomes, are as discussed above.

In various embodiments, the at least one liposome in the liposomal formulation may be a multilamellar vesicle or a unilamellar vesicle. The at least one liposome may have a size of 30 nm to 2 μm, 30 nm to 1 μm, 100 nm to 1 μm, 500 nm to 1 μm, 30 nm to 100 nm, 30 nm to 500 nm, 100 nm to 500 nm or any other sizes or ranges falling within these ranges. In various embodiments, the size may be 100 nm to 500 nm when the transition temperature of the one or more phospholipids may be 37° C. or more.

The liposomal formulation may be formed as an episcleral depot or an intrascleral depot according to the various embodiments.

The liposomal formulation may be used in treating or in the treatment of posterior ocular segment diseases comprising age-related macular degeneration (AMD), diabetic macular edema (DME), diabetic retinopathy etc. The liposomal formulation may also be used in treating or in the treatment of anterior ocular segment diseases comprising glaucoma, cataract, uveitis etc.

As disclosed earlier, the present disclosure further relates to a method of treating posterior and/or anterior ocular segment diseases by administering a liposomal formulation comprising one or more phospholipids, wherein the one or more phospholipids form at least one liposome each comprising at least one phospholipid bilayer, and wherein the one or more phospholipids may comprise or may consist of 1-palmitoyl-2-oleoyl-sn-glycero-3-phosphocholine (POPC), 1,2-dihexadecanoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 1,2-dipalmitoyl-3-trimethylammonium-propane (DPTAP), 1,2-dimyristoyl-3-trimethylammonium-propane (DMTAP), 1,2-dioleoyl-3-trimethylammonium-propane (DOTAP), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1-palmitoyl-2-oleoyl-sn-glycero-3-phosphoglycerol (POPG), hydrogenated soybean phosphatidylcholine (HSPC), 1-stearoyl-2-oleoyl-sn-glycero-3-phosphocholine (SOPC), or their combination thereof. The one or more phospholipids present in combination may be any positively or negatively charged phospholipids. Non-limiting examples of the combination may include POPC and DOTAP, POPC and POPG, DPTAP and POPG, DMTAP and POPG, DPPC and DPTAP, DPPC and DPPG, DMPC and DMTAP, or DMPC and DMPG.

Advantageously, due to the depot forming ability of the liposomal formulation, the liposomal formulation may be administered by subconjunctival injection which mitigates the risk of intravitreal injections.

The one or more phospholipids which may be used for the liposomal formulation administered may have a transition temperature of −18° C. to 55° C. or other any temperature or range within this specified range.

In various embodiments, the one or more phospholipids of the liposomal formulation administered may carry a net positive charge, a net negative charge or a net neutral charge. The net charge of the one or more phospholipids may depend on its combination with entities such as lipids, other molecule(s) or atom(s) etc. The method of treating ocular diseases using the present liposomal formulation is advantageously versatile as the charge of the phopholipids, and hence the liposomes, can be tailored based on the tissue surface or body (e.g. ocular) area as mentioned above.

In various embodiments, the one or more phospholipids of the liposomal formulation administered may comprise a saturated or an unsaturated phospholipid.

The at least one liposome comprising the saturated or unsaturated phospholipid of the liposomal formulation administered may or may not further comprise cholesterol.

The at least one liposome may form a multilamellar vesicle or a unilamellar vesicle before being administered. The multilamellar or unilamellar vesicle may have the configurations as described above. Advantageously, in the method of treating as disclosed herein, the rigidity or fluidity of the liposomal vesicle can be determined and altered before being administered in order to obtain the desired type of depots formed or to enhance penetration of the depots into the sclera.

In various embodiments, the at least one liposome of the liposomal formulation administered may have a size of 30 nm to 2 μm, 30 nm to 1 μm, 100 nm to 1 μm, 500 nm to 1 μm, 30 nm to 100 nm, 30 nm to 500 nm, 100 nm to 500 nm or any other sizes or ranges falling within these ranges. In various embodiments, the size may be 100 nm to 500 nm when the transition temperature of the one or more phospholipids of the liposomal formulation administered may be 37° C. or more.

The liposomal formulation may be formed as an episcleral depot or an intrascleral depot based on the needs of the present method which is used to treat various ocular diseases.

The ocular diseases may be posterior ocular segment diseases and/or anterior ocular segment diseases. The earlier may comprise age-related macular degeneration (AMD), diabetic macular edema (DME), diabetic retinopathy etc. while the latter may comprise glaucoma, cataract, uveitis etc.

The present disclosure also relates to the use of a particulate formulation comprising a plurality of poly(lactic-co-glycolic acid) (PLGA) particles in the manufacture of a medicament for the treatment of posterior and/or anterior ocular segment diseases, wherein the plurality of PLGA particles comprise PLGA microparticles and/or PLGA nanoparticles. The posterior ocular segment diseases may comprise age-related macular degeneration (AMD), diabetic macular edema (DME) or diabetic retinopathy etc. The anterior ocular segment diseases may comprise glaucoma, cataract or uveitis etc.

In various embodiments, the PLGA microparticles may be 10 μm to 200 μm, 50 μm to 100 μm, 100 μm to 150 μm or any other sizes falling within these ranges. The PLGA nanoparticles may be 150 nm to 500 nm, 150 nm to 250 nm, 250 nm to 500 nm or any other sizes falling within these ranges.

In various embodiments, the particulate formulation may be formed as an episcleral depot or an intrascleral depot.

The present disclosure also relates to a particulate formulation comprising a plurality of poly(lactic-co-glycolic acid) (PLGA) particles for use in the treatment of posterior and/or anterior ocular segment diseases, wherein the plurality of PLGA particles comprise PLGA microparticles and/or PLGA nanoparticles. The posterior and/or anterior ocular segment diseases may be diseases as mentioned above.

The size of the PLGA microparticles and PLGA nanoparticles may be as mentioned above.

The particulate formulation may be formed as an episcleral depot or an intrascleral depot.

The present disclosure further relates to a method of treating posterior and/or anterior ocular segment diseases by administering a particulate formulation comprising a plurality of poly(lactic-co-glycolic acid) (PLGA) particles, wherein the plurality of PLGA particles comprise PLGA microparticles and/or PLGA nanoparticles. The posterior and/or anterior ocular segment diseases may be diseases as mentioned above.

Advantageously, the particulate formulation may be administered by the less risky route of subconjunctival injection.

As mentioned above, the PLGA microparticles may be 10 μm to 200 μm, 50 μm to 100 μm, 100 μm to 150 μm or any other sizes falling within these ranges. The PLGA nanoparticles may be 150 nm to 500 nm, 150 nm to 250 nm, 250 nm to 500 nm or any other sizes falling within these ranges. The particulate formulation may be formed as an episcleral depot or an intrascleral depot.

While the uses of the liposomal and particulate formulation and the method of treating ocular diseases using the liposomal and particulate formulation as disclosed above are illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

### EXAMPLES

Amongst various sustained release carriers, liposomal microparticles and nanoparticles such as the liposomes disclosed herein are particularly attractive due to their biocompatibility, capability to deliver both hydrophobic and hydrophilic drugs, and their non-toxic nature. Advantageously, these liposomal particles and/or their liposomal particle formulations, such as liposomes of the present disclosure, are able to form and act as subconjunctival depots on administration without being cleared away by circulation or lymphatic drainage. These depots may be either episcleral or intrascleral while sustaining the release of any encapsulated drug(s) over long periods. In order to demonstrate tunability of the depot systems, studies based on their size, charge and chemical properties are demonstrated in the following examples. The above advantages may also apply to particulate formulations derived from PLGA microparticles and/or nanoparticles.

### Example 1: Types of Liposomes

The liposomes for the liposomal microparticle and/or nanoparticle formulations as described herein were made using the following lipids.

1-palmitoyl-2-oleoyl-sn-glycero-3-phosphocholine (POPC):

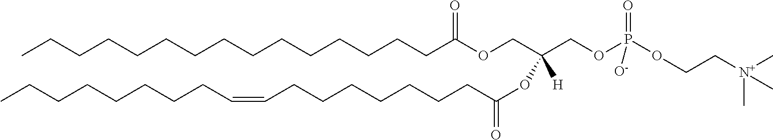 [SMILES: CCCCCCCC/C=C\CCCCCCCC(=O)O[C@H](COC(=O)CCCCCCCCCCCCCCC)COP(=O)([O-])OCC[N+](C)(C)C]

1,2-dihexadecanoyl-sn-glycero-3-phosphocholine (DPPC):

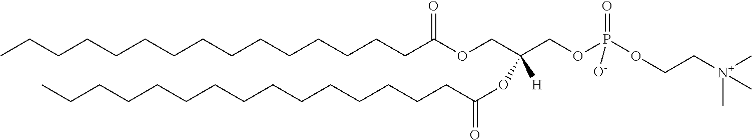 [SMILES: CCCCCCCCCCCCCCCC(=O)OC[C@H](COP(=O)([O-])OCC[N+](C)(C)C)OC(=O)CCCCCCCCCCCCCCC]

1,2-dioleoyl-sn-glycero-3-phosphocholine (DOPC):

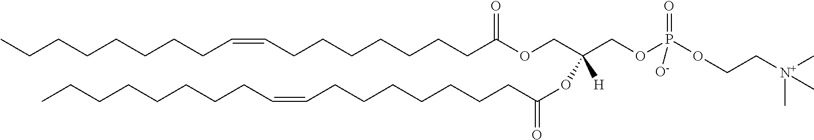 [SMILES: CCCCCCCC/C=C\CCCCCCCC(=O)OC[C@H](COP(=O)([O-])OCC[N+](C)(C)C)OC(=O)CCCCCCC/C=C\CCCCCCCC]

POPC combined with 1,2-dioleoyl-3-trimethylammonium-propane (DOTAP, a positively charged lipid). These two lipids co-exist in the liposomes maintaining their actual strutures. That is to say, the two lipids do not react to form a single chemical compound.

POPC combined with 1-palmitoyl-2-oleoyl-sn-glycero-3-phosphoglycerol (POPG, a negatively charged lipid). These two lipids co-exist in the liposomes maintaining their actual strutures. That is to say, the two lipids do not react to form a single chemical compound.

### Example 2: Production of Liposomes

Liposomes were produced by the thin-film hydration technique. The lipids were first dissolved in a chloroform-methanol mixture. The solvent was then evaporated, forming a thin film which was subsequently hydrated to spontaneously form multilamellar vesicles (MLVs). The MLVs were then extruded sequentially through polycarbonate filters of different sizes fitted in a bench top extruder, (Northern Lipids Inc Canada) to obtain large unilamellar vesicles (LUVs) of desired sizes.

### Example 3: Effect of Liposomes' Size

The liposomes made from lipids with lower transition temperature, which were more fluid in structure (i.e. bilayer structure of the liposomes allows better diffusion in and out of the liposome), are able to act as intrascleral depots. Smaller sized liposomes of less than about 1 μm exhibited a larger degree of transport inside the sclera compared to larger liposomes. Particularly, smaller liposomes of about 73 nm were observed to have a higher degree of transport compared to the 94 nm sized liposomes. Even larger liposomes of about 1 μm or more acted as episcleral depots on the scleral surface as they transport to a smaller extent.

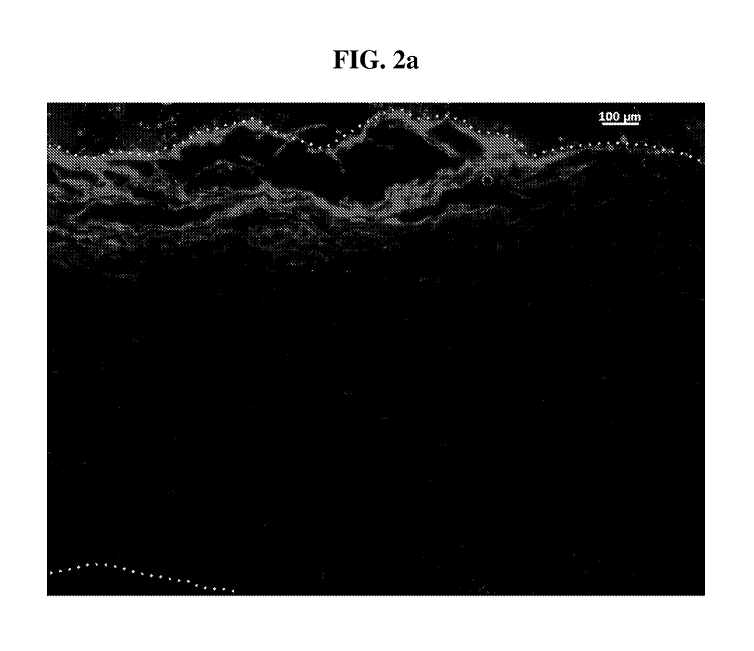

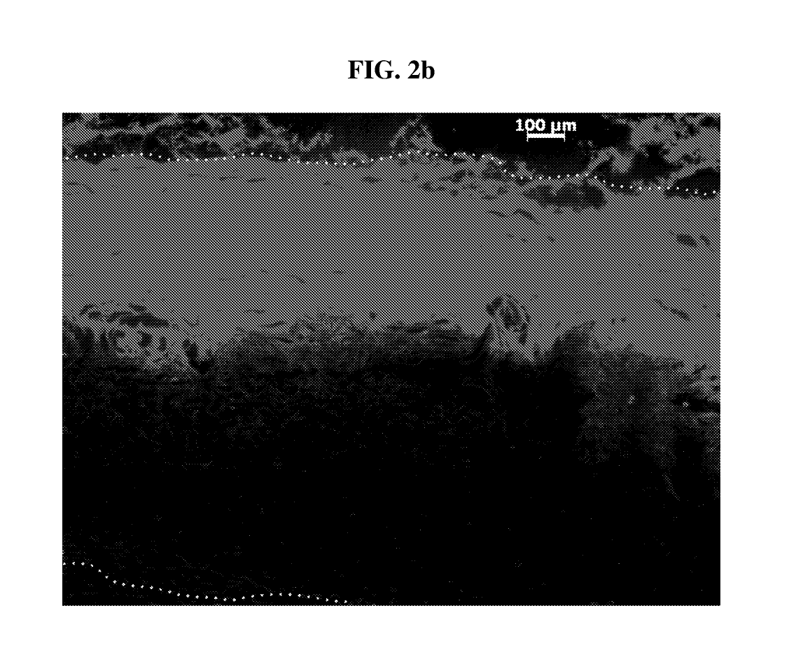

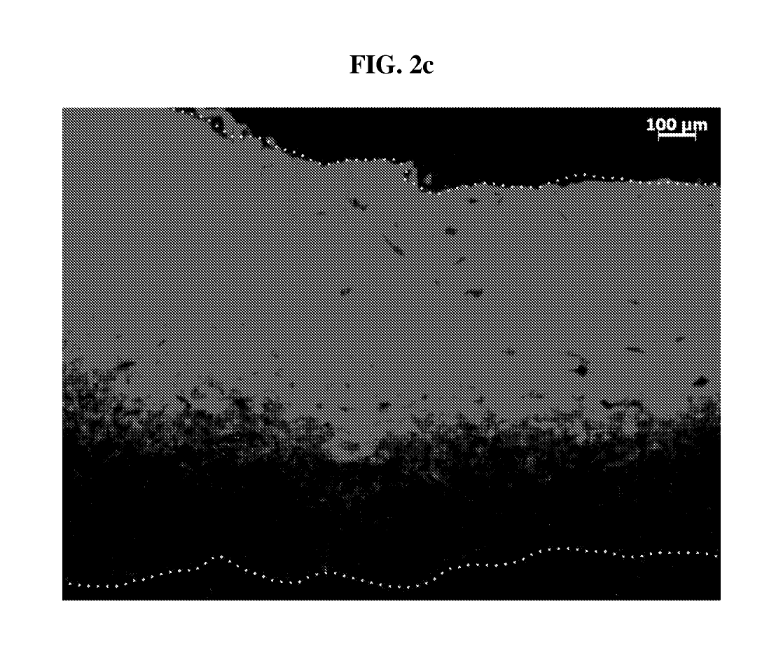

FIG. 2a to FIG. 2c demonstrate the effect of size of the POPC liposomes (transition temperature of −2° C.) on the depot forming ability. Fluorescent labelled liposomes were used for enhancing the observation. The effect of vesicle size on the distribution of liposomes inside the sclera can be observed. In FIG. 2a, MLVs of a larger size (about 1 μm) formed an episcleral depot without penetrating into the sclera. From FIG. 2b, it can be seen that LUVs of 90 nm formed an intrascleral depot which penetrated the sclera to some extent. As for FIG. 2c, it is observable that LUVs of 70 nm exhibited a larger intrascleral depot effect with higher penetration into the sclera. The liposomes were labelled with rhodamine-PE (red). The edges of the tissue are marked with a dotted line for clarity.

### Example 4: Effect of Bilayer Fluidity

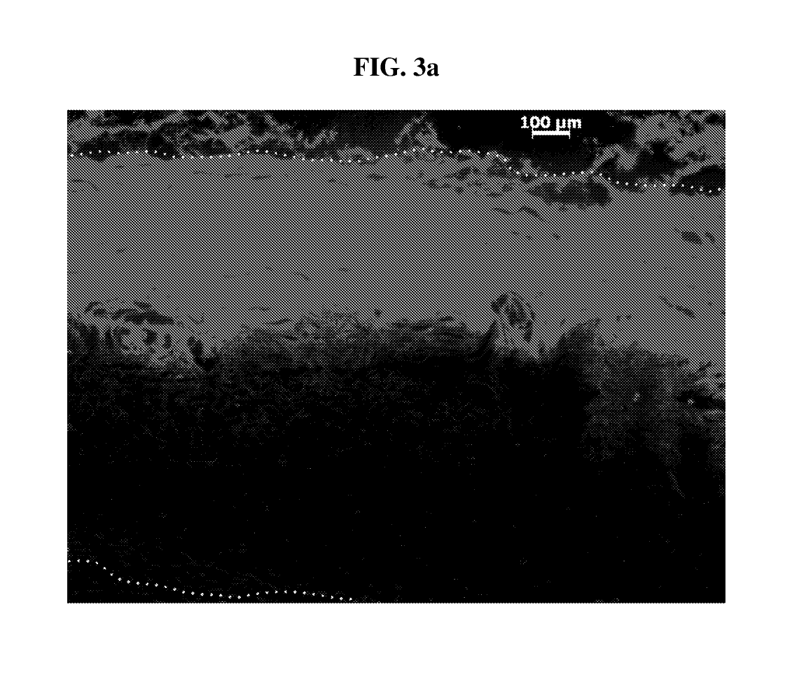

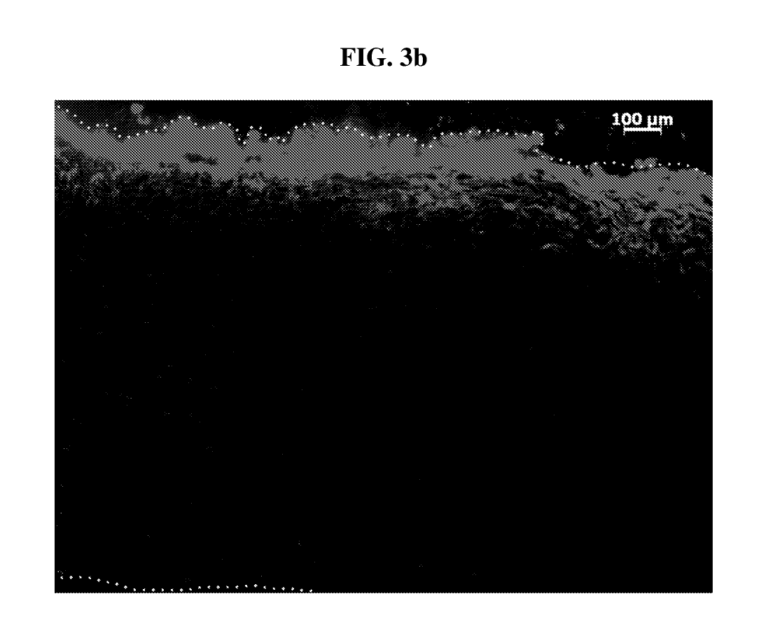

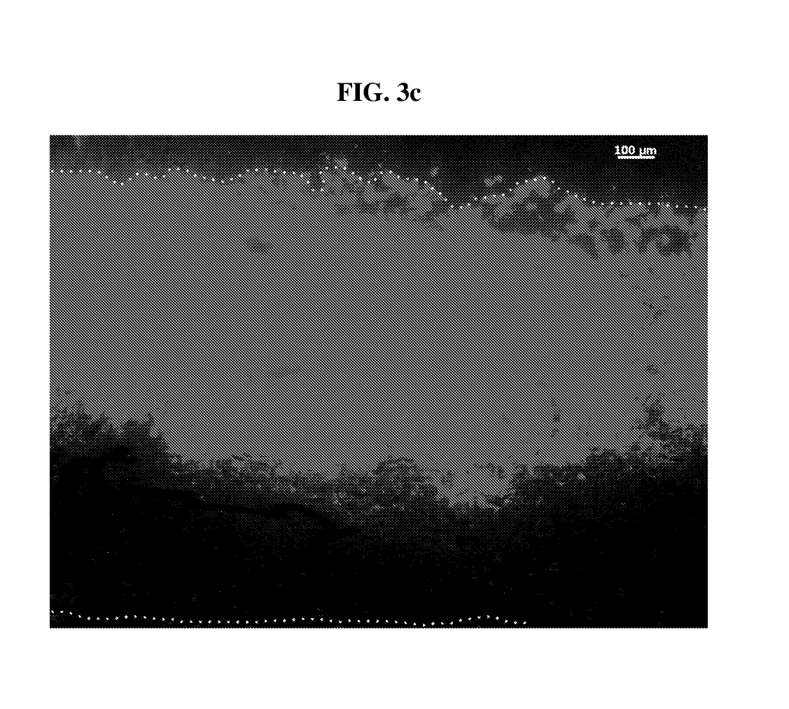

In comparison to the POPC liposomes (transition temperature of −2° C.) as illustrated above, liposomes of similar sizes having less bilayer fluidity such as DPPC (higher transition temperature of 41° C.) are found to form episcleral depots rather than intrascleral depots. This is also observed when compared to DOPC liposomes (transition temperature of −18° C.) of similar sizes. FIG. 3a to FIG. 3c show the effect of bilayer fluidity on the depot forming ability of the liposomal formulations. Accordingly, representative epifluorescent micrographs of the sclera post transport experiments wth fluorescent labelled POPC, DPPC and DOPC liposomes of a similar size are shown in FIG. 3a to FIG. 3c, respectively. Based on these figures, it can be observed that the POPC liposomes in FIG. 3a, which are more fluid, have the ability to act as intrascleral depots compared to the DPPC liposomes of FIG. 3b, which have a higher transition temperature and were found to transport less inside the sclera. The liposomes were labelled with rhodamine-PE (red) and the edges of the tissue are marked with a dotted line for clarity.

### Example 5: Effect of Liposomes' Charge

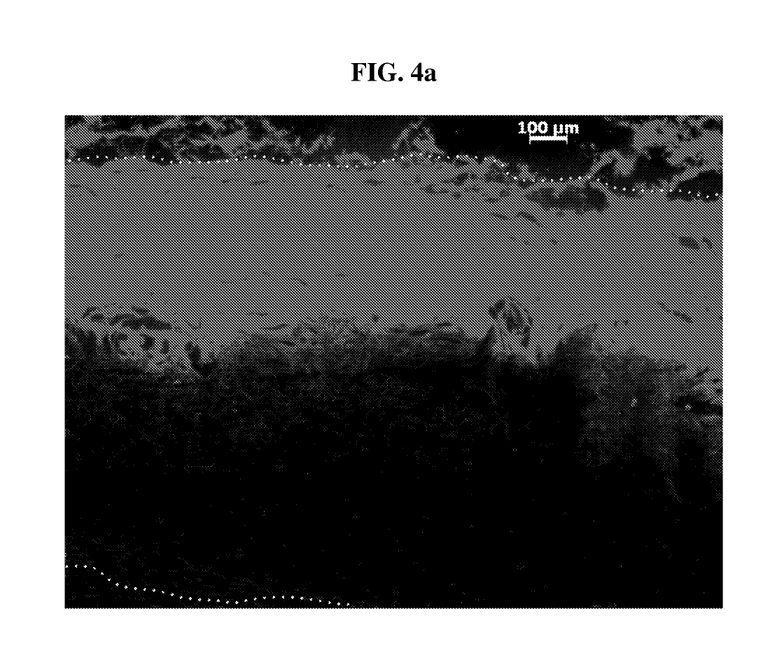

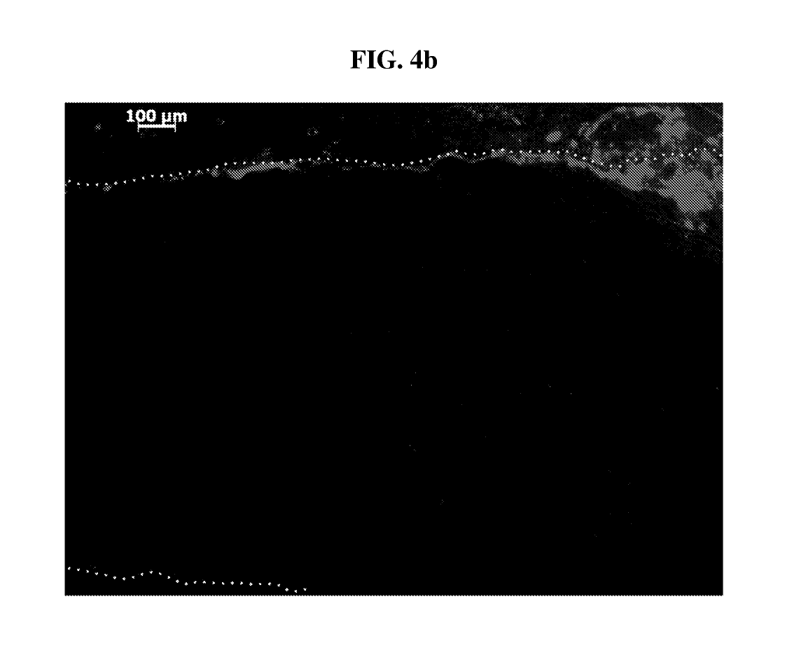

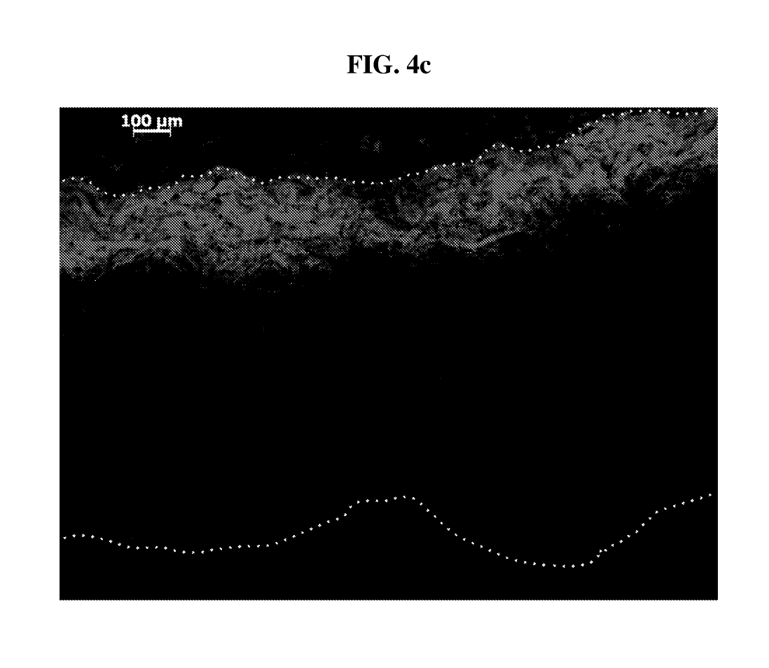

Charge of the liposomes is another parameter which can be used to tune the subconjunctival depot forming ability of the liposomal nanoparticles. As the sclera is negatively charged, positively charged liposomes are found to form episcleral depots rather than intrascleral depots. Negatively charged formulations were not expected to form in vivo subconjunctival depots as the charge repulsion may cause them to be rapidly cleared away by systemic circulation. FIG. 4a to FIG. 4c show the effect of charge on the depot forming ability of liposomal formulations. Accordingly, representative epifluorescent micrographs of the sclera post transport experiments with fluorescent labelled neutral, positive and negatively charged liposomes of the same size are shown in FIG. 4a to FIG. 4c, respectively. In FIG. 4a, POPC (neutral) liposomes formed intrascleral depots and demonstrated transport into the sclera. From FIG. 4b, POPC-DOTAP (positively charged) liposomes formed episcleral depots and stuck to the episcleral surface whereas POPC-POPG (negatively charged) liposomes of FIG. 4c were found to exhibit some transport into the sclera specifically for an ex vivo setup. However, in vivo, these negatively charged carriers are likely to be cleared away by circulation and unlikely form intrascleral or episcleral depots. The liposomes were labelled with rhodamine-PE (red) and the edges of the tissue are marked with a dotted line for clarity.

### Example 6: Addition/Presence of Cholesterol

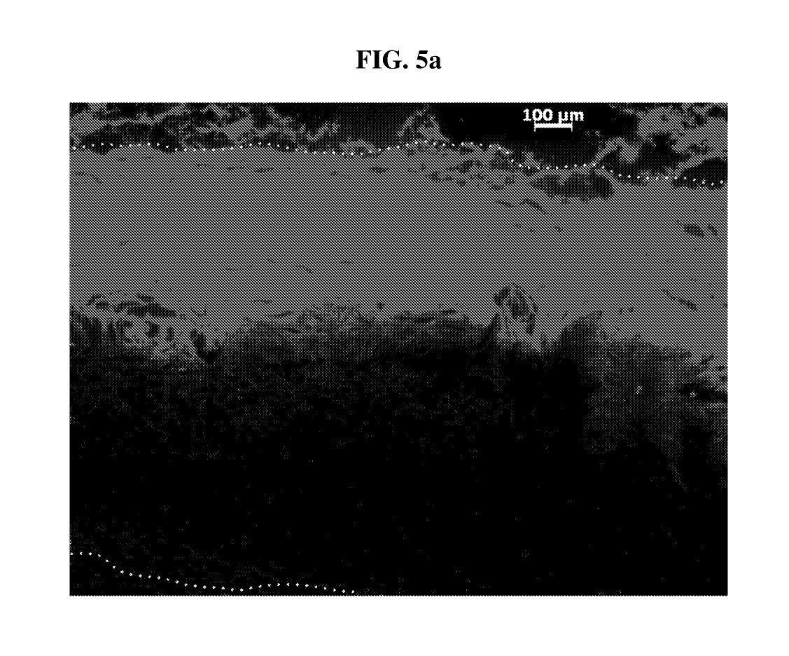

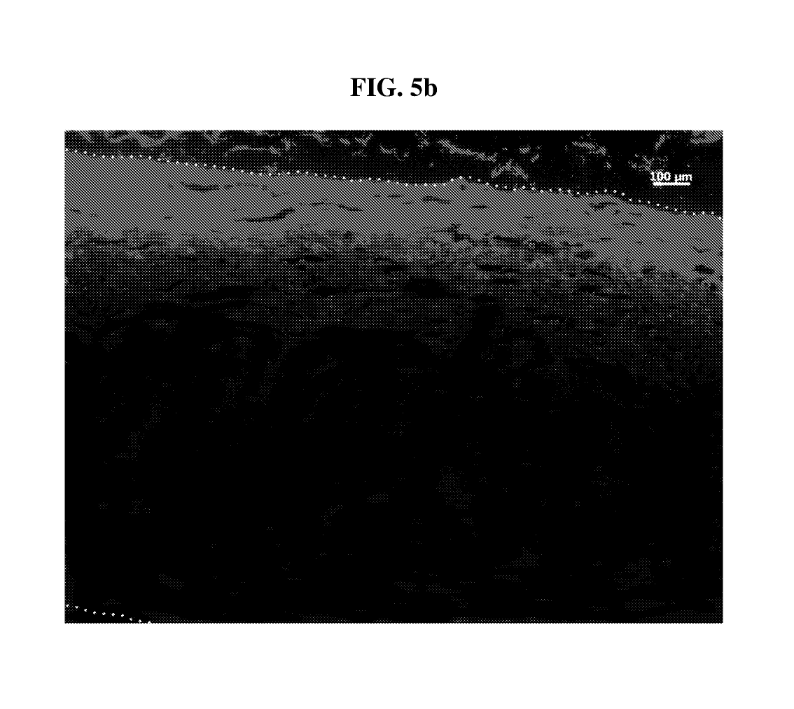

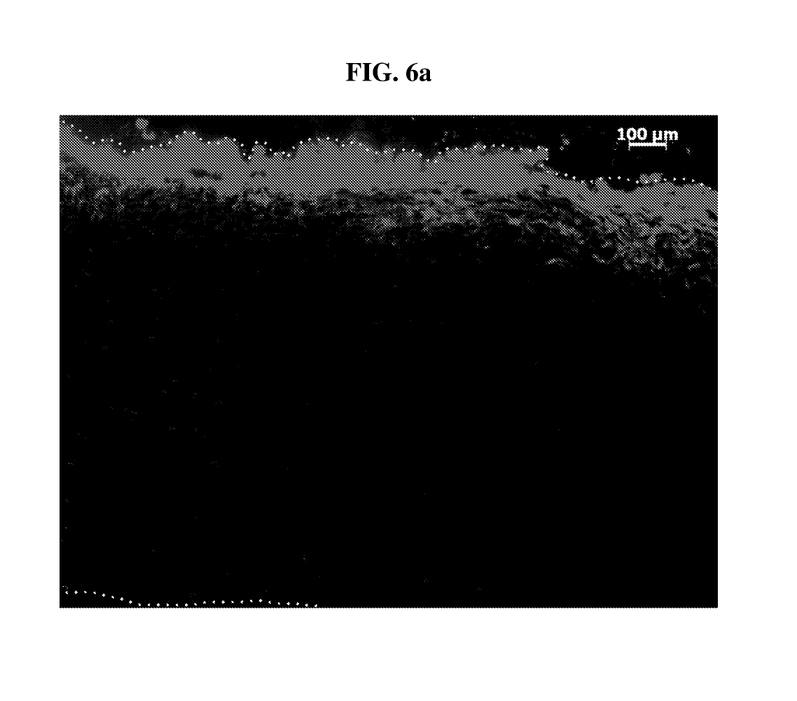

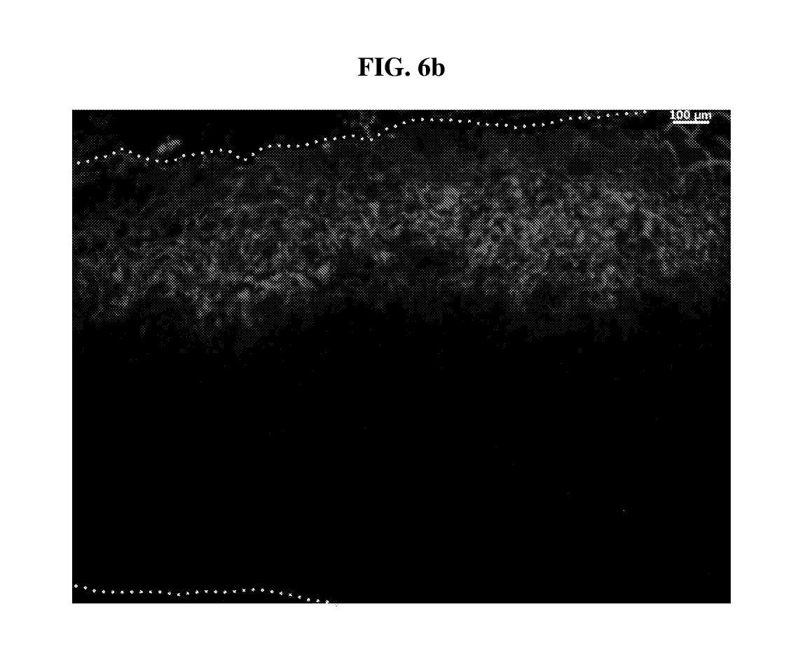

In addition, liposomes containing a cholesterol demonstrated depot forming ability, although this was to a lesser extent compared to bare liposome formulations as illustrated in the above examples. For unsaturated lipids such as POPC, addition of cholesterol causes the bilayer to become more rigid. This is reflected by the lesser degree of scleral penetration. On the other hand, in saturated lipids such as DPPC, the addition of cholesterol destabilizes the bilayer, making the bilayer leakier and the penetration of the liposomes containing cholesterol becomes higher than that of bare DPPC liposomes. This is depicted in FIG. 5a to FIG. 5b and FIG. 6a to FIG. 6b.

FIG. 5a and FIG. 5b show representative epifluorescent micrographs of the sclera post transport experiments with fluorescently labelled unsaturated liposomes without and with addition of cholesterol, respectively. In FIG. 5a, POPC (neutral) liposomes formed intrascleral depots and demonstrated transport into the sclera. Meanwhile, in FIG. 5b, POPC-Cholesterol (in a 80:20 molar ratio) were found to exhibit a lesser degree of transport into the sclera in an ex vivo setup. The liposomes were labelled with rhodamine-PE (red) and the edges of the tissue are marked with a dotted line for clarity.

FIG. 6a and FIG. 6b show representative epifluorescent micrographs of the sclera post transport experiments with fluorescently labelled saturated liposomes without and with addition of cholesterol, respectively. In FIG. 6a, DPPC (neutral) liposomes penetrate the sclera to a lesser extent compared to DPPC-Cholesterol (in a 80:20 molar ratio) of FIG. 6b. The liposomes were labelled with rhodamine-PE (red) and the edges of the tissue are marked with a dotted line for clarity.

### Example 7: Comparison with Poly(lactic-co-glycolic acid) Vehicles

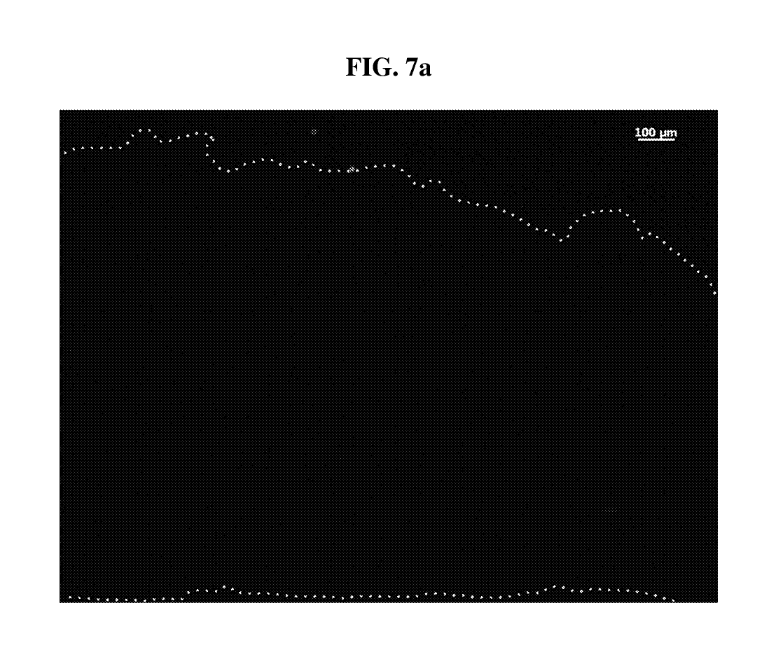

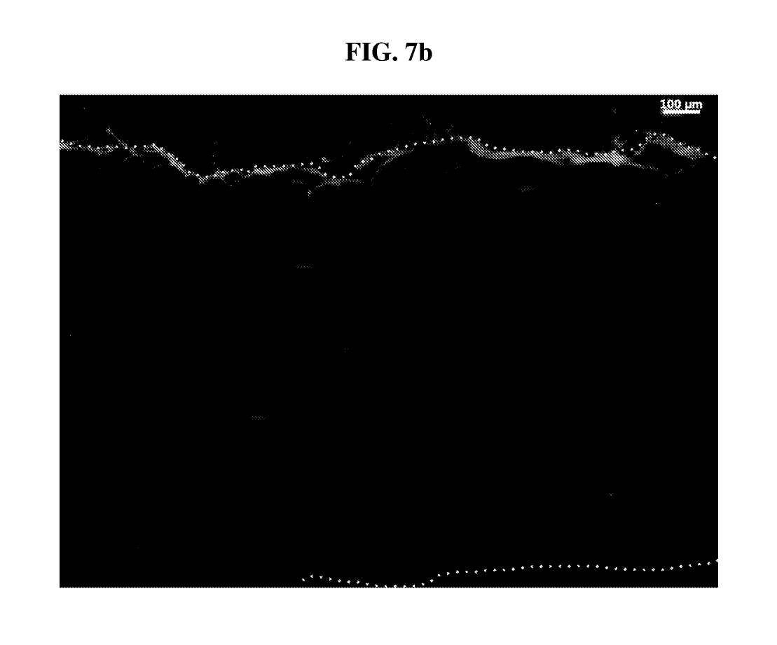

Poly(lactic-co-glycolic acid), also known as PLGA, is a FDA (i.e. the United States of America Food and Drug Administration) approved polymer with good biocompatibility and biodegradability properties. Several ocular drug delivery formulations have been developed using PLGA as a sustained release vehicle. Similar to distribution studies with liposomes as illustrated in the above examples, fluorescent tagged PLGA microparticles and nanoparticles were prepared and introduced in the donor chamber of the ex vivo setup. Generally, sizes above 1 μm are classified as microparticles in the context of the present disclosure. Their localization and trans-scleral transport were studied. From the episcleral micrograph of FIG. 7b, it can be observed that PLGA nanoparticles of approximately 200 nm showed episcleral localization after the ex vivo transport experiments. As for PLGA microparticles, due to their larger size, they may be retained in vivo although in the ex vivo setup of FIG. 7a, the PLGA microparticles were not observed to be retained on the episcleral surface.

### Example 8: Utility and Potential Commercial Applications

Based on the above examples, it can be summarized that a periocular, or more specifically, a subconjunctival injection of the liposomes or PLGA particle formulations as disclosed herein can lead to the formation of an episcleral or intrascleral depot of these carriers. The depots can be used to release drugs such as, but not limited to peptides, siRNA, proteins, in a sustained manner over time to reach the posterior segment of the eye.

From the examples and various embodiments as disclosed herein, nano-sized carriers with a positive charge forms episcleral depot(s) whereas nanocarriers that are neutral can localize in different places depending on fluidity (based on transition temperature) and size. More specifically, fluid carriers (in the case of liposomes with lower transition temperatures of less than 37° C.) form intrascleral depots while the more rigid ones (transition temperatures of 37° C. or more) form episcleral depots, especially those with sizes in the range of 100 nm to 500 nm.

Thus, the use of fluid and neutral nano-liposomes and micro-liposomes (size ranging 50 nm to 1000 nm) for depot based drug delivery to the back of the eye can be achieved via subconjuctival injection instead of intravitreal injection. Accordingly, the formulations, or more particularly the liposomes of the present disclosure, can be used to tune the subconjunctival depots based on the size, charge, chemical structure and rigidity of the nanocarriers. The application of these depot formulations comprising the ocular drugs are useful not only for posterior ocular segment diseases but may also be applied to the treatment of anterior segment diseases.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1-22. (canceled)

23. A method of treating posterior and/or anterior ocular segment diseases by administering a liposomal formulation comprising one or more phospholipids, wherein the one or more phospholipids form at least one liposome each comprising at least one phospholipid bilayer, and wherein the one or more phospholipids comprise 1-palmitoyl-2-oleoyl-sn-glycero-3-phosphocholine (POPC), 1,2-dihexadecanoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 1,2-dipalmitoyl-3-trimethylammonium-propane (DPTAP), 1,2-dimyristoyl-3-trimethylammonium-propane (DMTAP), 1,2-dioleoyl-3-trimethylammonium-propane (DOTAP), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1-palmitoyl-2-oleoyl-sn-glycero-3-phosphoglycerol (POPG), hydrogenated soybean phosphatidylcholine (HSPC), 1-stearoyl-2-oleoyl-sn-glycero-3-phosphocholine (SOPC), or their combination thereof.

24. The method according to claim 23, wherein the combination comprises POPC and DOTAP, POPC and POPG, DPTAP and POPG, DMTAP and POPG, DPPC and DPTAP, DPPC and DPPG, DMPC and DMTAP, or DMPC and DMPG.

25. The method according to claim 23, wherein the liposomal formulation is administered by subconjunctival injection.

26. The method according to claim 23, wherein the one or more phospholipids of the liposomal formulation administered carry a net positive charge, a net negative charge or a net neutral charge.

27. The method according to claim 23, wherein the one or more phospholipids of the liposomal formulation administered comprise a saturated or an unsaturated phospholipid.

28. The method according to claim 27, wherein the at least one liposome comprising the saturated or unsaturated phospholipid of the liposomal formulation administered further comprises cholesterol.

29. The method according to claim 27, wherein the at least one liposome comprising the saturated or unsaturated phospholipid of the liposomal formulation administered does not comprise cholesterol.

30. The method according to claim 28, wherein the at least one liposome forms a multilamellar vesicle or a unilamellar vesicle before being administered.

31. The method according to claim 23, wherein the at least one liposome of the liposomal formulation administered has a size of 30 nm to 2 μm.

32. The method according to claim 23, wherein the liposomal formulation forms an episcleral depot or an intrascleral depot.

33. The method according to claim 23, wherein the posterior ocular segment diseases comprise age-related macular degeneration (AMD), diabetic macular edema (DME) or diabetic retinopathy.

34. The method according to claim 23, wherein the anterior ocular segment diseases comprise glaucoma, cataract or uveitis.

35-53. (canceled)

54. The method according to claim 29, wherein the at least one liposome forms a multilamellar vesicle or a unilamellar vesicle before being administered.

55. A liposomal formulation comprising one or more phospholipids for use in the treatment of posterior and/or anterior ocular segment diseases, wherein the one or more phospholipids form at least one liposome each comprising at least one phospholipid bilayer, and wherein the one or more phospholipids comprise 1-palmitoyl-2-oleoyl-sn-glycero-3-phosphocholine (POPC), 1,2-dihexadecanoyl-sn-glycero-3-phosphocholine (DPPC), 1,2-dioleoyl-sn-glycero-3-phosphocholine (DOPC), 1,2-dimyristoyl-sn-glycero-3-phosphocholine (DMPC), 1,2-dipalmitoyl-3-trimethylammonium-propane (DPTAP), 1,2-dimyristoyl-3-trimethylammonium-propane (DMTAP), 1,2-dioleoyl-3-trimethylammonium-propane (DOTAP), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1-palmitoyl-2-oleoyl-sn-glycero-3-phosphoglycerol (POPG), hydrogenated soybean phosphatidylcholine (HSPC), 1-stearoyl-2-oleoyl-sn-glycero-3-phosphocholine (SOPC), or a combination thereof.
